(19) United States
(12) Patent Application Publication
(10) Pub. No.: US 2006/0104932 A1
(43) Pub. Date: May 18, 2006

(54) Exfoliating skin care product

(75) Inventor: Allen H. Rau, Cincinnati, OH (US)

Correspondence Address: MERCHANT & GOULD PC, P.O. BOX 2903, MINNEAPOLIS, MN, 55402-0903, US

(73) Assignee: PHYZZ, INC.

(21) Appl. No.: 11/274,333

(22) Filed: Nov. 16, 2005

Related U.S. Application Data
(60) Provisional application No. 60/628,205, filed on Nov. 17, 2004.

(51) Int. Cl. A61K 8/73

(52) U.S. Cl. 424/70.13; 424/401

(57) ABSTRACT

Exfoliating skin care products which rapidly dissolve in water, yet are non-irritating to the skin, are provided. These exfoliating skin care products comprise a water soluble particulate material of a large enough particle size such that the user of the exfoliating skin care product can tactilely feel the particulate material and an anhydrous liquid.

### CROSS REFERENCE TO RELATED CASES

This application claims the benefit of Provisional U.S. Patent Application No. 60/628,205, filed Nov. 17, 2004, which is incorporated by reference herein in its entirety.

### BACKGROUND OF THE INVENTION

1. Field of the Invention

Abrasive products that are designed to exfoliate the skin are well known. In general, solid particles of appropriate size that are insoluble in water are used to provide this function. Examples of materials used to provide this function are ground fruit (i.e. apricot, peach) seeds, and polyethylene beads. The problem with using insoluble materials is that it is risky for consumers to use them near their eyes. If they enter the eye, they cause irritation and can even scratch the cornea. Once they get in the eye, they are difficult to rinse and can cause extended periods of discomfort.

Water soluble scrub materials have been used. The most common of these are salt and sugar. Salt is not a particularly good choice for use in skin care products because, if the skin is compromised in any way (i.e. scratched or recently abraded by shaving), salt is very irritating. Also salt does not dissolve particularly quickly. Sugar is much less irritating than salt, however its dissolution rate is not particularly fast.

The aim of this invention is to provide an abrasive skin care product that utilizes a rapidly dissolving, water soluble, exfoliating material. The exfoliating material should be of sufficient size to provide its intended mild abrasive benefit and should also be capable of dissolving in water so as to not cause irritation or excess abrasion. Glucose polymers such as maltodextrins, corn syrup solids, starches and starch derivatives (such as hydroxypropylated starch, cationic starch, hydrogenated starch hydrosylate, sorbitol, maltitol, xylitol, polyglycitol and other sugar alcohols), when chosen with appropriate particle sizes, can provide this performance. Non-starch related materials, such as magnesium sulfate, magnesium carbonate, barium sulfate, aluminum sulfate, sodium tripolyphosphate and other forms of phosphate, can also provide this performance.

A further advantage of this invention is that glucose polymers are inherently less dense than alternate water soluble abrasives such as salt and sugar. This means that less weight of these polymers are needed to fill a given volume package. Even if polymers that cost more per pound than salt and sugar are chosen, the reduced weight needed to fill the package will often result in a finished product cost that is lower than salt or sugar based compositions.

2. Related Art

A search of the U.S. patent database revealed several prior art patents closely related to this invention.

U.S. Pat. No. 5,866,145 teaches a body polisher that utilizes sodium chloride and silicone oil as a two phase lotion that is designed to be gritty. This patent does not anticipate using materials other than sodium chloride to provide skin exfoliation.

U.S. Pat. No. 6,551,603 teaches a cosmetic salt scrub product that utilizes sodium sesquicarbonate as its scrubbing material. This patent does not anticipate anything other than sodium sesquicarbonate and sea salt as exfoliating materials.

U.S. Pat. No. 6,776,995 teaches souffle facial and body scrub products that are designed to exfoliate without being excessively abrasive or damaging to the skin. The only exfoliating materials used in this invention are coarse and fine salt.

U.S. Pat. Nos. 5,534,265 and 5,658,577 are closely related. They teach the inclusion of insoluble particles of various polymeric materials of small particle size (less than 75 microns) in cleansing compositions. This size particle is chosen so that the user does not tactilely perceive their presence in the product. The present invention aims to provide a tactile sensation from the particulate material.

### DETAILED DESCRIPTION OF THE INVENTION

This invention focuses on the use of water soluble materials that have large enough particle sizes to enable them to deliver a noticeable tactile sensation and to exfoliate the skin. Further, the particulate material is chosen so that it dissolves in water in a range of time. That is, slowly enough that it can deliver its exfoliating benefit but not so slowly that it doesn't completely dissolve during the usage period.

Materials that are particularly well suited for this purpose are glucose polymers such as maltodextrins, corn syrup solids, starches and starch derivatives (such as hydroxypropylated starch, cationic starch, hydrogenated starch hydrosylate, sorbitol, maltitol, xylitol, polyglycitol and other sugar alcohols) that have been agglomerated to build up their particle size. These materials are not soluble in oil, so they can be suspended in hydrophobic materials without risk of dissolution. Further, they do not dissolve instantaneously upon contact with warm water. Non-starch related materials, such as magnesium sulfate, magnesium carbonate, barium sulfate, aluminum sulfate, sodium tripolyphosphate and other forms of phosphate, can also provide this performance.

The following table illustrates the properties of some materials that work well:

| | Approx. Avg. | | Packed |
|---|---|---|---|
| | Particle Size | Dissolution | Density |
| Material | (microns) | Time (sec) | (g/cc) |
| Maltrin ® M700 | 400 | 27 | 0.13 |
| (Maltodextrin, 10 DE) | | | |
| Maltrin ® M500 | 250 | 45 | 0.34 |
| (Maltodextrin, 10 DE) | | | |
| Maltrin ® M585 | 235 | 30 | 0.40 |
| (Maltodextrin, 18 DE) | | | |
| Maltrin ® M600 | 235 | 45 | 0.40 |
| (Corn Syrup Solids, 20 DE) | | | |

Notes:
Maltrin ® products are commercially available from Grain Processing Corporation, Muscatine, IA. DE (Dextrose Equivalent) is a measure of glucose polymerization; Low DE indicates long polymer chains, High DE indicates greater quantities of glucose monomer (dextrose). By definition, starch has a DE of 0; dextrose's DE is 100.

For comparison purposes, comparable data on commonly used water soluble exfoliating materials are presented below:

| | Approx. Avg. | | Packed |
|---|---|---|---|
| | Particle Size | Dissolution | Density |
| Material | (microns) | Time (sec) | (g/cc) |
| Table Sugar (sucrose) | 250 | >120 | 1.05 |
| Table Salt (sodium chloride) | 250 | >120 | 1.40 |

For the purposes of the above tables, dissolution time is defined as the length of time it takes 5 grams of test material to dissolve in 300 ml of 40° C. (±2° C.) water being stirred with a magnetic stir bar at speed setting “4” on a Thermolyne Nuova II stir plate.

It should be noted that particles below 75 microns are generally regarded as being too fine to offer tactile sensation. They are certainly too small to provide exfoliation benefits. Scrub type products are generally formulated with a solid abrasive material that is either suspended in or covered with a liquid. In products where the abrasive material is water soluble (such as salt or sugar), an anhydrous liquid is chosen to prevent dissolution of the solid. Many anhydrous liquids are available. Typical materials include mineral oils, vegetable oils, silicone oils, fatty acid alcohols and esters.

Surfactants may be included in the anhydrous liquid to aid in rinsing or create foam. The particular surfactant(s) chosen will depend on the effect that is desired. Typical surfactants that can be used include Polysorbate 80, Polysorbate 85, PEG-40 Sorbitan Peroleate, glyceryl esters, various ethoxylated fatty alcohols, sodium lauryl sulfate, sodium lauryl sulfoacetate, cocamidopropyl betaine, and others. The surfactant preparations used in the invention should be substantially anhydrous and free of alcohols of 1-6 carbon atoms.

Fragrance and color can be added to the product at the formulator's discretion. Such fragrances and colors would be well known to one of skill in the skin care art.

The following formulations illustrate the invention and how it compares to products based on salt and sugar.

| | Inventive Formulas | | | Comparative Formulas | |
|---|---|---|---|---|---|
| | (% w/w) | | | (% w/w) | |
| Material | 1 | 2 | 3 | A | B |
| Sunflower Oil | 72.7 | 52.2 | 52.2 | 29.2 | 20.7 |
| Polysorbate 85 | 4.0 | 3.0 | 3.0 | 2.0 | 1.5 |
| PEG-40 | 4.0 | 3.0 | 3.0 | 2.0 | 1.5 |
| Sorbitan | | | | | |
| Peroleate | | | | | |
| Preservative | 0.3 | 0.3 | 0.3 | 0.3 | 0.3 |
| Fragrance | 4.0 | 4.0 | 4.0 | 4.0 | 4.0 |
| Maltrin ® M700 | 15.0 | | | | |
| Maltrin ® M500 | | 37.5 | | | |
| Maltrin ® M600 | | | 37.5 | | |
| Table Sugar | | | | 62.5 | |
| Table Salt | | | | | 72.0 |
| Total | 100.0 | 100.0 | 100.0 | 100.0 | 100.0 |

It should be noted that each of these formulations are designed to use an amount of oil that would wet and cover the solids in a way that provides a spreadable, cosmetically aesthetically acceptable product. In the cases of the inventive products, the solid materials remain fairly well suspended. That is, there is no visible oil layer on top or on the bottom of them. In the cases of the comparable formulas, the solids settle very rapidly and oil “pools” on the surface. In general, the ratio of anhydrous liquid to particulate matter is unrestricted. A preferred range for the products of the invention is about 5-70% particulate matter.

The products listed above were used in the following manner: The skin was wet with warm water first. Then the product was applied and massaged on the skin. Additional water was added to dilute and rinse the product. It was found that all products yielded a gritty feel while being massaged on the skin. The inventive products, however, quickly broke down and rinsed cleanly as they were diluted and rinsed. The comparative products continued to feel gritty and were difficult to completely rinse.

It is understood that skilled formulators can modify these products to optimize the benefits that they wish to deliver. For instance, the type and quantity of surfactant can be chosen to deliver more or less creamy lather or leave more or less moisturizing materials on the skin. Also, polymeric materials and various emollients can be incorporated to deliver particular skin feels. Of course, colorants and many types of fragrance can also be utilized with this technology.

The fact that the inventive products are easier to rinse than currently available technology is one of their advantages. Another benefit of the relatively rapid dissolution of the solids is that there is less chance for injury if a consumer would get a particle in his/her eyes. This type of incident is particularly common when insoluble particles such as ground apricot seeds, strawberry seeds or polyethylene beads are used as scrubbers.

The inventive products are also superior to salt based products because they are non-irritating to the skin. It is well known that when salt contacts a recently abraded or physically compromised area of a person's skin, they will experience a burning sensation. This may be of particular concern to people who wish to use an exfoliating product after shaving their legs or face.

A further advantage of the invention over salt based scrub products is economic. Although many glucose polymer products are more expensive (per pound) than salt or sugar, the fact that they can be used a lower levels than the salt or sugar, as demonstrated in the above formulas, means that the resulting product may be less costly than salt or sugar based products. This is benefit is further amplified by the fact that the glucose polymer are less dense than salt or sugar. Thus fewer pounds of them are needed to fill a given size container. Thus, a particular volume container will require less weight of product to fill, thus becoming more economical.

I claim:

1. An exfoliating skin care product comprising:
a water soluble particulate material of a large enough particle size such that the user of the exfoliating skin care product can tactilely feel the particulate material, wherein the water soluble particulate material dissolves in water between about 20 seconds and about 90 seconds at 40° C.; and
an anhydrous liquid.

2. The exfoliating skin care product of claim 1, wherein said water soluble particulate material is an agglomerated glucose polymer.

3. The exfoliating skin care product of claim 2, wherein said agglomerated glucose polymer is selected from the group consisting of agglomerated maltodextrins, agglomerated corn syrup solids, agglomerated starch and agglomerated corn syrup solids.

4. The exfoliating skin care product of claim 1, wherein said water soluble particulate material has a particle size of 75 microns or greater.

5. The exfoliating skin care product of claim 1, wherein the anhydrous liquid is selected from the group consisting of mineral oils, vegetable oils, silicone oils, fatty acid alcohols and esters.

6. The exfoliating skin care product of claim 1, further comprising a surfactant.

7. The exfoliating skin care product of claim 6, wherein said surfactant is selected from the group consisting of Polysorbate 80, Polysorbate 85, PEG-40 Sorbitan Peroleate, glyceryl esters, various ethoxylated fatty alcohols, sodium lauryl sulfate, sodium lauryl sulfoacetate, and cocamidopropyl betaine.

8. The exfoliating skin care product of claim 1, further comprising a fragrance.

9. The exfoliating skin care product of claim 1, further comprising a color.

10. The exfoliating skin care product of claim 1, further comprising a polymeric material.

11. The exfoliating skin care product of claim 1 further comprising an emollient.
